(19) United States
(12) Patent Application Publication
(10) Pub. No.: US 2025/0354101 A1
(43) Pub. Date: Nov. 20, 2025

(54) BIONIC ORGAN DEVICE

(71) Applicant: DARWIN PRECISIONS CORPORATION, Hsinchu County (TW)

(72) Inventors: Yu-Hsin Wu, Hsinchu County (TW); Ting-Chieh Yang, Hsinchu County (TW); Pang-Chun Liu, Hsinchu County (TW); Jui-Hua Lin, Hsinchu County (TW)

(21) Appl. No.: 19/026,510

(22) Filed: Jan. 17, 2025

(30) Foreign Application Priority Data
May 15, 2024 (TW) 113117986

(51) Int. Cl. C12M 1/12; C12M 1/00

(52) U.S. Cl. CPC C12M 25/04; C12M 23/34

(57) ABSTRACT

A bionic organ device comprising an organ chip. The organ chip comprises a first body, a second body, and a porous membrane, where the porous membrane is disposed between the first and second bodies, and the porous membrane comprises a polymer fiber mesh and a hydrophilic polymer coating. The polymer fiber mesh has a plurality of pores, and the hydrophilic polymer coating coats the polymer fiber mesh.

### FIELD OF THE INVENTION

The present invention relates to a bionic technology, specifically to a bionic organ device that can simulate the microenvironment of a living organism.

### BACKGROUND OF THE INVENTION

Traditional cell culture models cannot reflect the complicated physiological functions of biological tissues and organs. Animal experiments have drawbacks such as long cycles, high costs, and difficulties in directly predicting the true responses of organisms. Organ chips mimic key functions of organs in living organisms, reconstructing the physiological environment of organs in the body, simulating the structure(s), microenvironment, and physiological functions of organs in living organisms. They also offer accurate parameter control, along with advantages of miniaturization, integration, high efficiency, and reduced costs. To simulate the stretching and contraction of organ cells, porous hydrogel membranes are adapted in current organ chips. Porous hydrogel membranes can be used for cell attachment and are generally prepared through a film-flipping process; however, during the membrane detachment process, damage due to insufficient strength is prone to occur. In addition, when porous hydrogel membranes are used in organ chips and subjected to significant stretching and contraction, they are also prone to damage.

### SUMMARY OF THE INVENTION

The invention provides a bionic organ device which can be used to stimulate dynamic microenvironment of organs, tissues, cells, etc., and has good reliability.

The bionic organ device provided by the invention comprises an organ chip. The organ chip comprises a first body, a second body, and a porous membrane. The porous membrane is disposed between the first body and the second body and comprises a polymer material fiber mesh and a hydrophilic polymer material coating. The polymer material fiber mesh has a plurality of pores, and the hydrophilic polymer material coating coats the polymer material fiber mesh.

The present invention is beneficial to more precisely control the breathability of the porous membrane, and to enhance the strength of the porous membrane by adapting the polymer material fiber mesh and the hydrophilic polymer material coating, and better meets the operational requirements of the organ chip. Reliability of the organ chip is also further improved and the service life of the same is extended due to the porous membrane.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic three-dimensional view of a bionic organ device according to an embodiment of the invention;

FIG. 2 shows a decomposition of FIG. 1;

FIG. 3 is a schematic cross-sectional view taken along line A-A″ in FIG. 1;

FIG. 4 is a schematic top view of a porous membrane according to an embodiment of the invention;

FIG. 5 is a schematic cross-sectional view taken along line B-B″ in FIG. 4; and

FIG. 6 is a schematic three-dimensional view of a polymer material fiber mesh according to an embodiment of the invention.

### DETAILED DESCRIPTION OF PREFERRED EMBODIMENTS

The foregoing and other technical contents and other features and advantages of the present invention will be clearly presented from the following detailed description of a preferred embodiment in cooperation with the accompanying drawings. Directional terms mentioned in the following examples are only used to describe directions referring to the attached drawings. Therefore, the directional terms used are for illustration and not for limitation. In addition, terms such as “first” and “second” involved in the description or claims are merely used for naming the elements or distinguishing different embodiments or ranges rather than limiting the upper limit or lower limit of the quantity of the elements.

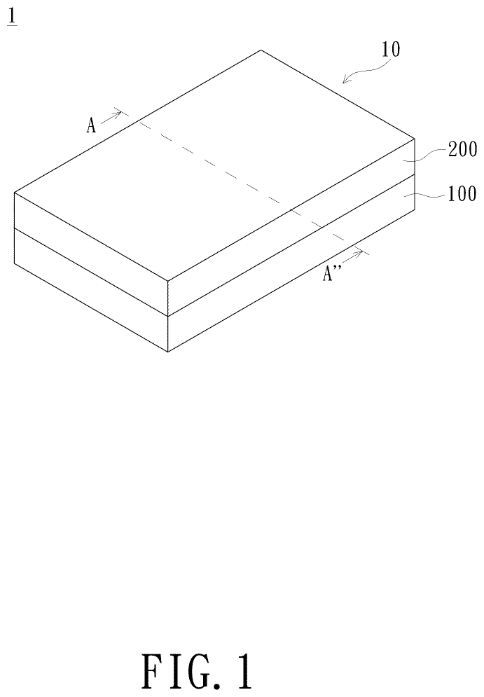

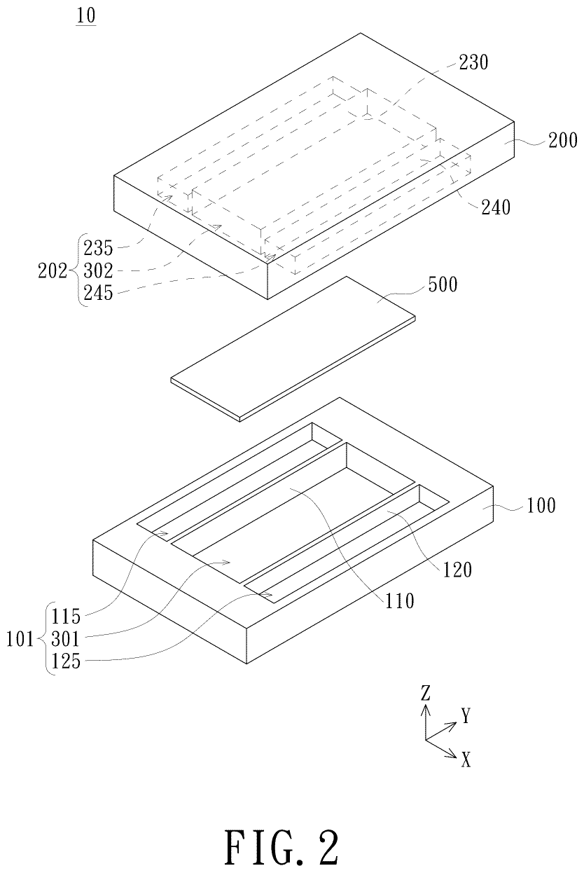

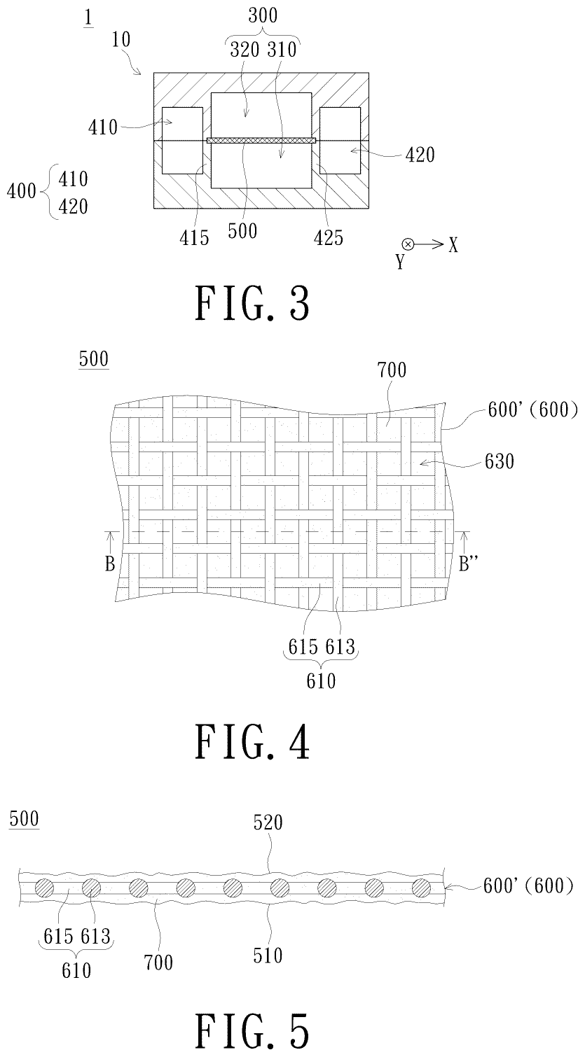

FIG. 1 is a schematic three-dimensional view of a bionic organ device according to an embodiment of the invention, FIG. 2 shows a decomposition of FIG. 1, and FIG. 3 is a schematic cross-sectional view taken along line A-A″ in FIG. 1. As shown in FIGS. 1-3, the bionic organ device 1 in the embodiment of the present invention includes an organ chip 10. The organ chip 10 includes a first body 100, a second body 200, and a porous membrane 500. The porous membrane 500 is disposed between the first body 100 and the second body 200 and forms a flow channel system 300 with the first body 100 and the second body 200. Further, the first body 100 and the second body 200 each may have a cavity-like structure to have an accommodating space and an opening. The opening of the first body 100 and the opening of the second body 200 face each other and together form the flow channel system 300 with the porous membrane 500. The flow channel system 300 includes a first flow channel 310 located between the first body 100 and the porous membrane 500 and a second flow channel 320 located between the second body 200 and the porous membrane 500. The first flow channel 310 and/or the second flow channel 320 allows at least one fluid (not shown) to pass through or stay therein. The flow channel system 300 may further include an inlet/outlet port (not shown) communicated with the first flow channel 310 and/or the second flow channel 320. The inlet/outlet port can be arranged in any known manner on the organ chip 10. For example, the inlet/outlet port may be formed on the first body 100 and/or the second body 200 and further enable a communication between the interior and exterior of the organ chip 10.

The porous membrane 500 can be used as a cell attachment membrane for cells to attach to the surface of the membrane. The porous membrane 500 may be bonded to the surface of the first body 100 facing the second body 200 and the surface of the second body 200 facing the first body 100. The bonding can be achieved through one or more known methods, such as thermal pressing, welding, adhesive bonding, or other means that can realize the bonding. In the embodiment of the invention, the materials of the first body 100 and the second body 200 are preferably plastics, such as polycarbonate (PC), polyethylene terephthalate (PET), polypropylene (PP), polyvinyl chloride (PVC), or polydimethylsiloxane (PDMS), but are not limited to these. The means of realizing the bonding may vary depending on the materials of the first body 100 and the second body 200. For example, when the materials of the first body 100 and the second body 200 are polycarbonate, the bonding between the first body 100 and the second body 200 and the porous membrane 500 can be achieved through means such as thermal pressing. On the other hand, when the materials of the first body 100 and the second body 200 are polydimethylsiloxane, it is preferable to perform surface treatment on the bonding surfaces of the first body 100 and the second body 200 and the bonding area of the porous membrane 500 before bonding, and subsequently enhances the bonding strength through means such as baking.

FIG. 4 is a schematic top view of a porous membrane according to an embodiment of the invention. FIG. 5 is a schematic cross-sectional view taken along line B-B″ in FIG. 4. As shown in FIGS. 4 and 5, in the embodiment of the invention, the porous membrane 500 is composed of a polymer material fiber mesh 600 and a hydrophilic polymer material coating 700. As shown in FIG. 5, the hydrophilic polymer material coating 700 coats the polymer material fiber mesh 600 and forms a first membrane surface 510 and a second membrane surface 520 of the porous membrane 500. In the embodiment of the invention, when the porous membrane 500 forms the flow channel system 300 with the first body 110 and the second body 120, the first membrane surface 510 may be located in the first flow channel 310, the second membrane surface 520 may be located in the second flow channel 320, and both of the first membrane surface 510 and the second membrane surface 520 are adapted for cell attachment. When there is a fluid in the first flow channel 310 or the second flow channel 320, the fluid can contact either the first membrane surface 510 or the second membrane surface 520.

The polymer material fiber mesh 600 can be woven from polymer material fibers or from yarns 610 made of polymer material fibers. In the embodiment of the invention, the polymer material fibers are preferably synthetic fibers, such as polyester fibers, polyamide (nylon) fibers, or polyacrylonitrile (acrylic) fibers, but are not limited thereto. Further, the polymer material fiber mesh 600 can be made of a single type of fiber or multiple types of fibers. For example, multiple types of fibers can be blended to form the yarns 610, which are then woven into a mesh, or various yarns 610 made of different fibers are interwoven into a mesh. The yarns 610 spun from polymer material fibers is preferably with appropriate strength, and therefore the woven mesh has tensile strength. In some embodiments of the invention, the polymer material fiber mesh 600 is woven from yarns 610 spun from polyester fibers.

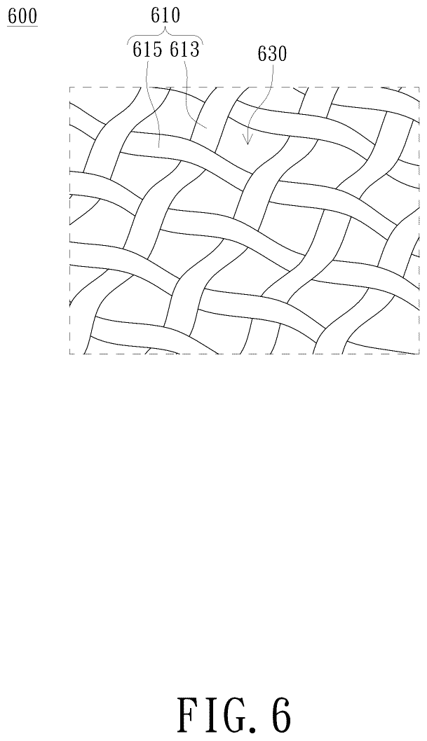

Specifically, the yarns 610 can be interlaced in both directions to form a mesh. The aforementioned two directions can be perpendicular to each other, such as the warp and weft directions. The yarns 610 in the warp direction can be referred to as warp yarns 613, and the yarns 610 in the weft direction can be referred to as weft yarns 615. In this embodiment of the invention, the yarns 610 used for weaving can have a diameter of, for example, 10 to 500 μm, such as 50 μm, 100 μm, 150 μm, 200 μm, 250 μm, 300 μm, 350 μm, 400 μm, or 450 μm. As shown in FIGS. 4 and 6, the adjacent yarns 610 in the mesh 600′ are preferably not tightly spaced, thus forming a plurality of pores 630 in the mesh 600′. The pores 630 ensure the breathability of the porous membrane 500, where the total area of the pores 630 preferably occupies a specific percentage of the area of the mesh 600′. The aforementioned percentage is also known as the opening rate. In addition, the size of the pores 630 can vary depending on the tightness of the weave and the diameter of the yarns 610. In the embodiment of the invention, the average diameter of the pores 630 can be, for example, 0.1 to 5 μm, such as 0.5 μm, 1 μm, 1.5 μm, 2 μm, 2.5 μm, 3 μm, 3.5 μm, 4 μm, or 4.5 μm. The pore size can be designed according to the cells to be attached and the experimental requirements. For example, an appropriately sized pore can prevent certain molecules secreted by cells from unexpectedly passing through the pores 630 and moving between the two sides of the porous membrane 500.

The yarns 610 may be woven in other ways besides interlacing in the warp and weft directions to form a mesh. For example, the yarns 610 are not limited to interlacing in only two directions but can be woven in multiple directions to form the mesh 600′. In fact, any weaving method commonly seen in fabrics or textiles can be suitably applied to the present invention. Regardless of the weaving method used, the mesh 600′ preferably has a specific opening rate as described above. In a preferred embodiment of the invention, the opening rate is, for example, 10 to 30%, such as 10%, 15%, 20%, 25%, or 30%. The opening rate can be related to the breathability of the mesh.

In general, the mesh 600′ formed by interlacing the plurality of yarns 610 in two or more directions on a plane can be substantially equivalent to the polymer material fiber mesh 600 and can be used to form the porous membrane 500 with the hydrophilic polymer material, but not limited thereto. For example, the polymer material fiber mesh 600 can further include a plurality of meshes 600′, such as a plurality of meshes 600′ arranged in overlapping layers. Additionally, in some embodiments, the plurality of yarns 610 can be woven in multiple directions across different dimensions to form the mesh 600′. For example, a three-dimensional mesh can be formed by interlacing the yarns 610 in three or more directions across three dimensions.

The polymer material fiber mesh 600 is coated with a hydrophilic polymer material to form the porous membrane 500 having the hydrophilic polymer coating 700. In other words, the hydrophilic polymer coating 700 of the porous membrane 500 can be directly formed on the polymer material fiber mesh 600. In the embodiment of the invention, the hydrophilic polymer material is a biocompatible material that is soft, flexible, and has breathability or porosity. The pores in the hydrophilic polymer material generally have sizes measured in nanometers (nm), covering ranges such as several nanometers, tens of nanometers, or hundreds of nanometers. The hydrophilic polymer material may contain functional groups such as —OH, —CONH, —CONH₂, —COOH, —SO₃H, and can be formed through chemical or physical cross-linking of monomers having these functional groups. In the embodiment of the invention, the hydrophilic polymer material can be either natural or synthetic. Natural materials include polysaccharides such as cellulose, starch, hyaluronic acid, alginate, chitosan, and polypeptides such as collagen, poly-L-lysine, and poly-L-glutamic acid, but are not limited to these. Synthetic materials include polymers such as polyacrylic acid, polymethacrylic acid, and polyacrylamide.

In the preferred embodiments of the invention, the hydrophilic polymer material is a hydrogel. The hydrogel of the hydrophilic polymer material can be formed, for example, by cross-linking acrylic acid or its derivatives, acrylamide or its derivatives, hydroxyethyl methacrylate or its derivatives, but is not limited thereto. The hydrogel can be molded through thermosetting or thermoplastic methods. For example, the hydrogel in a sol or fluid state can be molded by heating or cooling within a mold. In the embodiment of the invention, the hydrogel can be used to coat the polymer material fiber mesh 600 in its sol state and then be shaped into the hydrophilic polymer coating 700 and the porous membrane 500 through heating or cooling.

In the embodiments of the invention, the hydrophilic polymer material is not limited to fully covering the polymer material fiber mesh 600. For example, the hydrophilic polymer coating 700 may not completely cover the surrounding portions of the polymer material fiber mesh 600. In principle, the surface portions of the porous membrane 500 exposed to the flow channel system 300, such as the first membrane surface 510 and the second membrane surface 520, are parts of the hydrophilic polymer coating 700. Other areas, such as the bonding area of the porous membrane 500 with the first body 100 and the second body 200, may not have a distribution of the hydrophilic polymer coating 700. In the embodiments of the invention, the porous membrane 500 can have a thickness of, for example, several tens of micrometers, where the thickness is composed of the thickness of the polymer material fiber mesh 600 and the thickness of the hydrophilic polymer coating 700. For example, the porous membrane 500 may have a thickness of approximately 20 to 35 μm. In some embodiments of the invention, the thickness of the porous membrane 500 is 27 to 33 μm, and is preferably 30 μm.

When the porous membrane 500 is used as the cell adhesion membrane, cells can be attached to the first membrane surface 510 and/or the second membrane surface 520 and can be further cultured in the aforedescribed fluid. The same or different kinds of cells can be attached to the first membrane surface 510 and the second membrane surface 520, and the fluids in the first flow channel 310 and the second flow channel 320 can vary according to the kinds of cells. For example, the first membrane surface 510 can be for the attachment of alveolar epithelial cells, the second membrane surface 520 can be for the attachment of microvascular endothelial cells, the first flow channel 310 can be supplied with oxygen-containing gas, and the second flow channel 320 can be supplied with culture fluid.

As described above, the porous membrane 500 has pores 630 and the hydrophilic polymer material has breathability or porosity, accordingly, small molecules on both sides of the porous membrane 500 have the opportunity to pass through it and move between the first flow channel 310 and the second flow channel 320, which allows the organ chip 10 to simulate the phenomena of organs, tissues, or cells in a dynamic microenvironment within a living organism. Further, since the polymer material fiber mesh 600 of the porous membrane 500 has tensile strength and the hydrophilic polymer material is extensible, the porous membrane 500 gains strength and flexibility, which allow it capable of repeated stretching, contraction, or even greater degrees of stretching and contraction without damage. In some embodiments of the invention, the stretching and contraction of the porous membrane 500 can correspond to a change in length thereof in one direction. For example, an amount of the change in length in the direction can be 0.5% to 10% of a length of the porous membrane 500 in that direction, such as 0.5%, 1%, 2%, 2.5%, 3%, 3.5%, 4%, 4.5%, 5%, 5.5%, 6%, 6.5%, 7%, 7.5%, 8%, 8.5%, 9%, 9.5%, or 10%. For example, when the length of the porous membrane 500 in a first direction X is 10 millimeters, a 0.5% change in length would be stretching to 10.05 millimeters or contracting to 9.95 millimeters in the first direction X, while a 10% change in length would be stretching to 11 millimeters or contracting to 9 millimeters in the first direction X.

The stretching and contraction of the porous membrane 500 can be achieved through one or more known methods, and the invention does not limit this. In the preferred embodiments of the invention, the polymer material fiber mesh 600 of the porous membrane 500 can have a tensile strength of, for example, 500 to 1000 MPa, thereby providing the porous membrane 500 with higher tensile strength. For example, the tensile strength can be greater than 50 MPa and up to nearly 1000 MPa, such as 100 to 950 MPa, and more preferably 500 to 950 MPa. Therefore, regardless of the method used to achieve the stretching and contraction of the porous membrane 500, the porous membrane 500 in the embodiments of the invention, due to its strength and flexibility, is adapted to multiple stretchings and contractions without damage. In some embodiments of the invention, the stretching and contraction of the porous membrane 500 can be achieved, for example, through a vacuum system, as described below.

In the embodiment of the present invention, the organ chip 10 further comprises a vacuum system 400, which can also be composed of the first body 100 and the second body 200, as described below. As shown in FIGS. 2 and 3, the first body 100 further comprises a first inner wall 110 and a second inner wall 120. The first inner wall 110 and the second inner wall 120 divide the accommodating space 101 of the first body 100 into a plurality of chambers which include a first half-chamber 115 on one side and a second half-chamber 125 on the other side, and a first flow channel preparation space 301 between the two half-chambers 115 and 125. The second body 200 comprises a third inner wall 230 and a fourth inner wall 240. The third inner wall 230 and the fourth inner wall 240 divide the cavity 202 of the second body 200 into a plurality of chambers, including a third half-chamber 235 and a fourth half-chamber 245 respectively on two opposite sides, and a second flow channel preparation space 302 between the two half-chambers 235 and 245.

The first inner wall 110 and the second inner wall 120 described above are aligned respectively with the third inner wall 230 and the fourth inner wall 240. Accordingly, when the first body 100 and the second body 200 are assembled, the first flow channel preparation space 301, the second flow channel preparation space 302, and the porous membrane 500 together form the flow channel system 300. In addition, a first vacuum chamber 410 and a second vacuum chamber 420 are formed on two opposite sides of the flow channel system 300 along the first direction X, where the first vacuum chamber 410 is formed by the first half-chamber 115 and the third half-chamber 235, and the second vacuum chamber 420 is formed by the second half-chamber 125 and the fourth half-chamber 245. The first vacuum chamber 410 and the second vacuum chamber 420 constitute the vacuum system 400 of the organ chip 10. The vacuum system 400, especially the first vacuum chamber 410 thereof, is separated from the flow channel system 300 via the first wall 415, and the second vacuum chamber 420 is separated from the flow channel system 300 via the second wall 425. The first wall 415 is composed of the first inner wall 110 and the third inner wall 230, and the second wall 425 is composed of the second inner wall 120 and the fourth inner wall 240. The first inner wall 110 and the third inner wall 230, as well as the second inner wall 120 and the fourth inner wall 240, can be joined together through the preciously described bonding process to form the first wall 415 and the second wall 425.

The vacuum system 400 can be used to actuate the porous membrane 500 in the flow channel system 300. Further speaking, the walls between the vacuum system 400 and the flow channel system 300, such as the first wall 415 and the second wall 425, are preferably designed to be elastically deformable, and preferably, they can bend substantially in the first direction X. The elastic deformation of the first wall 415 and the second wall 425 can in turn actuate the porous membrane 500, causing it to stretch, contract, or return to its original state. For example, when the first vacuum chamber 410 and the second vacuum chamber 420 are near a vacuum state, the first wall 415 and the second wall 425 can bend towards the direction of the vacuum chambers 410 and 420 due to the pressure difference between the vacuum system 400 and the flow channel system 300. This bending can then actuate the porous membrane 500, causing it to stretch towards the first vacuum chamber 410 and the second vacuum chamber 420. Conversely, when the pressure difference decreases, the first wall 415 and the second wall 425 can return to their original state, and the porous membrane 500 can also return to its original state. Thus, when the porous membrane 500 serves as the cell adhesion membrane, the porous membrane 500 can, for example, be stretched and recovered back and forth, to let the cells attached thereto be able to perform simulation of phenomena such as stretching, contracting, and recovery in a dynamic microenvironment. In the preferred embodiments of the present invention, the porous membrane 500 can be stretched or contracted multiple times in the first direction X by an amount relative to its original length within 0.5% to 10% without damage.

The configurations of the first vacuum chamber 410 and the second vacuum chamber 420 in the aforedescribed vacuum system 400, as well as the configurations of the first wall 415 and the second wall 425, are merely exemplary and do not impose limitations on the present invention. It is understood that regardless of the configuration of the vacuum system 400 in the organ chip 10 for achieving the stretching, contracting, and/or recovery of the porous membrane 500, the porous membrane 500 is adapted to repeated stretching, contracting, and recovering without significant damage due to its strength and deformability. Therefore, the organ chip 10 in the embodiments of the present invention has high reliability.

The bionic organ device 1 of the embodiments of the present invention may further comprise components related to the functionality of the organ chip 10. For example, the bionic organ device 1 may comprise input/output tubing and fluid supply modules, which can communicate with the flow channel system 300 through the input/output ports (not shown), and are used to supply fluids (not shown) to flow through or reside within the flow channel system 300. The bionic organ device 1 may also comprise a vacuum extraction module (not shown), which can generate a pressure differential between the vacuum system 400 and the flow channel system 300, thereby actuating the porous membrane 500 to stretch, contract, and recover. Due to its strength and deformability, the porous membrane 500 is suitable for repeated stretching, contracting, and recovering without significant damage. Therefore, the bionic organ device 1 in the embodiments of the present invention has high reliability.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A bionic organ device, comprising an organ chip, the organ chip comprising a first body, a second body, and a porous membrane, wherein:
the porous membrane is disposed between the first body and the second body and comprises a polymer material fiber mesh and a hydrophilic polymer material coating, the polymer material fiber mesh has a plurality of pores, and the hydrophilic polymer material coating coats the polymer material fiber mesh.

2. The bionic organ device according to claim 1, wherein the porous membrane has a tensile strength of 50 to 1000 MPa.

3. The bionic organ device according to claim 1, wherein the polymer material fiber mesh is further formed by weaving a plurality of yarns, the plurality of yarns are made of polymer material fibers, and each of the yarns has a diameter ranging from 10 to 500 μm.

4. The bionic organ device according to claim 1, wherein each of the pores of the polymer material fiber mesh has an average pore size, and the average pore size ranges from 0.1 to 5 μm.

5. The bionic organ device according to claim 1, wherein the plurality of pores constitute an opening rate of the polymer material fiber mesh, and the opening rate ranges from 10% to 30%.

6. The bionic organ device according to claim 1, wherein the polymer material fiber mesh are made of polymer material fibers, and the polymer material fibers are further artificial fibers.

7. The bionic organ device according to claim 1, wherein the polymer material fiber mesh are made of polymer material fibers, and the polymer material fibers include polyester fibers.

8. The bionic organ device according to claim 1, wherein the polymer material fiber mesh comprises at least one mesh formed by interlacing a plurality of warp yarns and a plurality of weft yarns.

9. The bionic organ device according to claim 1, wherein the polymer material fiber mesh has a tensile strength of 500 to 1000 MPa.

10. The bionic organ device according to claim 1, wherein the porous membrane has deformability and is adapted to change in length in one direction.

11. The bionic organ device according to claim 10, wherein an amount of the change in length is 0.5% to 10% of a length of the porous membrane in the direction.

12. The bionic organ device according to claim 1, wherein the hydrophilic polymer material coating is further formed by a hydrophilic polymer material on the polymer material fiber mesh, and the hydrophilic polymer material includes a hydrogel.

13. A porous membrane adapted to use in an organ chip, and the porous membrane comprising:
a polymer material fiber mesh, woven from polymer material fibers and having a plurality of pores; and
a hydrophilic polymer material coating, formed by coating the polymer material fiber mesh with a hydrophilic polymer material.
